(19) United States
(12) Patent Application Publication
(10) Pub. No.: US 2023/0123521 A1
(43) Pub. Date: Apr. 20, 2023

(54) FOOT HYGIENE DEVICE

(71) Applicant: PUMIPOD CLEVEBE SPÓLKA Z OGRANICZONA ODPOWIEDZIALNOSCIA, Jasionka (PL)

(72) Inventor: Lukasz NICPON, Lipie (PL)

(21) Appl. No.: 17/909,424

(22) PCT Filed: Feb. 26, 2021
(86) PCT No.: PCT/IB2021/051631

§ 371 Date: Sep. 5, 2022

(30) Foreign Application Priority Data
Mar. 4, 2020 (PL) P.433144

(51) Int. Cl. A61B 17/54

(52) U.S. Cl. CPC A61B 17/54

(57) ABSTRACT

A foot hygiene device, intended for the removal of calluses and coarse skin comprising a body made of plastic with an abrasive, characterized in that the body has an arched upper part (1) detachably connected to the abrasive (5) which is covered with abrasive material, and has a lower part (2), parallel to the ground, with at least one, separated in it, pressing plane (4), located outside the outline of the upper arched part (1).

### TECHNICAL FIELD

The object of the present invention is a foot hygiene device, intended for the removal of calluses and coarse skin accumulated on the feet.

### BACKGROUND ART

Patent specification PL228606 discloses a foot hygiene device, intended for the removal of calluses and coarse skin, which comprises an arched body made of plastic provided with ribs at the bottom, which in the upper part has a central recess with side walls parallel to the longitudinal axis of the device and with a front wall semicircular in outline, the walls are angled towards the body and connected with it by gentle curves and, moreover, the body has a recess at the front in which it is located a lever of a suction cup made of elastomer and attached from the bottom to the body, and additionally the upper part of the body is permanently covered with an abrasive having grain size in the range of 100 to 1500 μm.

The disadvantage of this solution is that the body is covered with abrasive permanently. The abrasive wears out fairly quickly, i.e. it crumbles and dulls. When the abrasive is worn out, the device is no longer functional, cannot be reused and must be thrown away.

The technical problem that required solution was to construct a foot hygiene device eliminating the disadvantage mentioned above, reducing the time of use of the device until the moment, when the abrasive covering the surface of the body is worn out.

### DISCLOSURE OF THE INVENTION

This problem is solved by a foot hygiene device, intended for the removal of calluses and coarse skin, which comprises a body made of plastic with an abrasive, characterized in that the body has an arched upper part detachably connected to the abrasive which is covered with abrasive material, and has a lower part, parallel to the ground, with at least one—separated in it—pressing plane, located outside the outline of the upper arched part.

Preferably in the upper part the body has ergonomic shape, what means, that it is profiled such that its vertical cross-section is close to the heel curvature.

Preferably the upper part has a groove and protrusions, and the abrasive has positioning holes for protrusions.

Preferably the protrusions have form of circles and/or crescents.

Preferably the abrasive element is connected with the upper arched part of the body indirectly via intermediary element.

Preferably the intermediary element has a form of a self-adhesive tape covered with small hooks that cling to a second cloth-covered tape, commonly known as Velcro, or a form of double-sided semi-adhesive tape.

Preferably the abrasive is an abrasive material with a grain size from 30.2 μm to 269 μm.

Preferably the abrasive is abrasive foil or sandpaper or abrasive cloth.

Preferably on the underside of the lower part, the body has recesses.

Preferably on the underside of the lower part, the body has self-locking pads, made of anti-slip and/or increased adhesion material.

Preferably the self-locking pads are silicone inserts having thickness of 1 mm to 3 mm.

Preferably in the side wall of the arched upper part, the body has a hole.

The main advantage of the invention is that the abrasive element is detachably connected with the body. Thanks to this solution, the abrasive element is replaceable and the device is suitable for multiple use.

Another advantage of the solution according to the invention is that:
in the upper part the body has ergonomic shape, profiled such that its cross section is close to the heel curvature, what facilitates abrasion of skin in this region;
in the lower part the body has at least one plane parallel to the ground, located outside the outline of the upper part of the body, what enables the device to be pressed to the ground during use, making it more stable and safer;
in the upper arched part the body has a groove and protrusions, in which groove the upper part of the abrasive is inserted, the said part having positioning holes, in which the protrusions are embedded, which additionally stabilizes the abrasive element and prevents uncontrolled detachment of the abrasive element from the body;
the device has self-locking pads to prevent it from sliding on slippery surfaces;
it is suitable for removing the skin from the feet when taking a shower, practically without bending down.

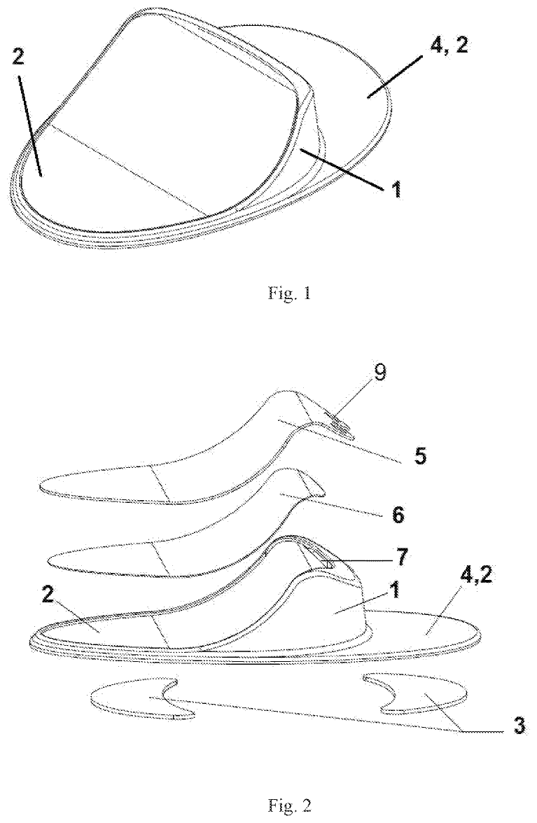

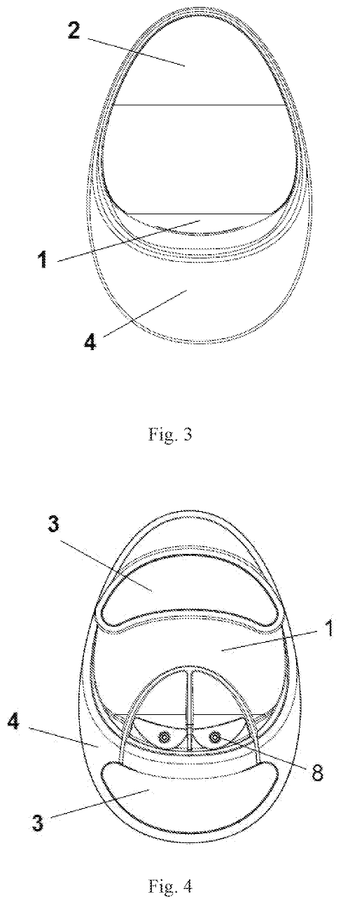

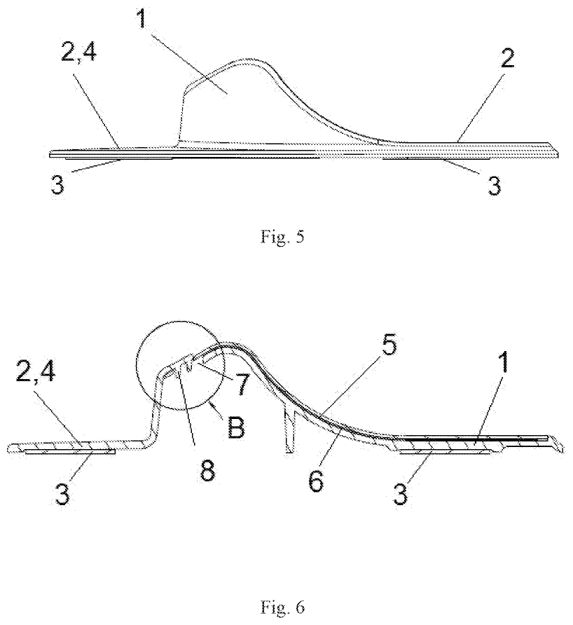

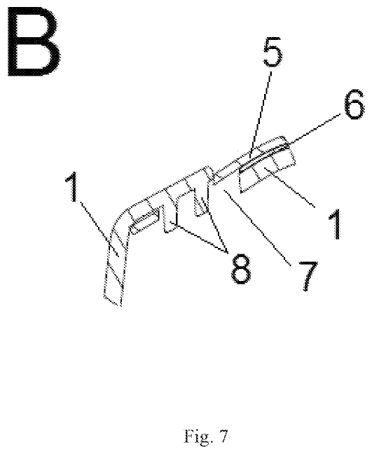

The subject of the invention is disclosed in the example below at the drawings, where FIG. 1 shows the device in isometric projection, FIG. 2 shows the device in isometric projection, FIG. 3 shows the device in a top view, FIG. 4 shows the device in a bottom view, FIG. 5 shows the device in a side view, FIG. 6 shows the device in a side view and a vertical cross-section, FIG. 7 shows the enlarged detail marked in FIG. 6.

### EXAMPLES

### Example 1

A foot hygiene device has a body made of plastic. The body has an upper part 1 arched towards the top of the device and a lower part 2 which is parallel to the ground.

On the underside of the lower part 2, the body has recesses for self-locking pads 3 to prevent the device from sliding on slippery surfaces. Preferably, the self-locking pads 3 are in the form of silicone inserts having thickness of 1 mm to 3 mm, what however does not exclude other solutions.

It the lower part 2, the body has at least one separated pressing plane 4, also parallel to the ground. The pressing plane 4 is located outside the outline of the upper arched part 1. The pressing plane 4 helps to press the device to the ground during use. One foot presses the tool to the ground via the pressing plane 4, and the other foot performs rubbing action to remove calluses and thickened skin. After performing the activity with one of the feet, the position of the feet on the device changes and the activity is repeated.

In the upper part 1 the body has ergonomic shape, what means, that it is profiled such that its vertical cross section is close to the heel curvature. It facilitates abrasion of skin in this region.

In the upper arched part 1, an abrasive 5 is attached, which is an abrasive material having grain size from 30.2 μm to 269 μm. (P60 to P500 according to FEPA scale), preferably in the form of abrasive foil, what however does not exclude other solutions like use of sand paper or abrasive cloth.

The abrasive 5 is shaped accordingly to the body. The abrasive 5 also has ergonomic shape, to fit the upper arched part 1 of the body to which it is attached. The ergonomic shape of the abrasive element 5 can be achieved by any technique, for example by folding the abrasive in several axes, that are parallel to each other.

The abrasive 5 is detachably connected to the arched upper part 1 of the body. Preferably, the abrasive element 5 is indirectly connected to the arched upper part 1 via intermediary element 6 in the form of a self-adhesive tape covered with small hooks that cling to the second cloth-covered tape, together forming an easy-to-unfasten and fasten connection, commonly known as Velcro.

The Velcro fastening solution allows to easily detach the abrasive 5 from the arched upper part 1, thanks to which, it can be replaced with a new one, while the body is used many times, contrary to the solutions existing on the market, that after the wear out of the abrasive 5 are not suitable for reuse.

In order to additionally prevent the abrasive element 5 from uncontrolled detaching from the body, in the arched upper part 1 there is a groove 7 and protrusions 8. Into the groove 7 the top part of the abrasive element 5 is inserted, having positioning holes 9, that make the abrasive 5 more stable during use of the device. In the positioning holes 9 protrusions 8 are embedded, having shape that facilitates this, preferably circles or crescents.

### List of Components:

1—upper arched part of the body
2—bottom part of the body
3—self-locking pads
4—plane in the bottom part 2
5—abrasive
6—intermediary element between part 1 and 5
7—groove in part 1
8—protrusions in part 1
9—holes in element 5

### Example 2

The device as in the example 1, where the abrasive 5 is detachably connected with the arched upper part 1 via intermediary element 6 in the form of a double-sided adhesive tape.

### Example 3

The device as in the example 1 or the example 2, wherein the body has a hole in the side wall of the arched upper portion 1, allowing the device to be easily levered with a toe if it is sucked to the ground during use. Thanks to this, there is no need to bend down, which is an advantage of the solution and increases the usefulness of the device.

1. A foot hygiene device, intended for removal of calluses and coarse skin, which comprises a body made of plastic with an abrasive, characterized in that the body has an arched upper part (1), has ergonomic shape, what means that is profiled such that its vertical cross-section is close to the heel curvature and (1) has a groove (7) and protrusions (8), and the abrasive (5) has positioning holes (9) for protrusions (8), additionally the upper part (1) is detachably connected to the abrasive (5) which is covered with abrasive material, and has a lower part (2), parallel to the ground, with at least one—separated in it—pressing plane (4), located outside the outline of the upper arched part (1), the abrasive (5) is connected with upper arched part (1) of the body indirectly via an intermediary element (6), and the intermediary element (6) has a form of a self-adhesive tape coverered with small hooks that cling to a second cloth-covered tape, commonly known as Velcro, or in the form of double—sided semi-adhesive tape.

2. The device according to claim 1, characterized in that the protrusions (6) have form of circles and/or crescents.

3. The device according to claim 1, characterized in that the abrasive element (5) is an abrasive with a grain size from 30.2 pm to 269.

4. The device according to claim 3, characterized in that the abrasive (5) is abrasive foil or sandpaper or abrasive cloth. circles.

5. The device according to claim 1, characterized in that on the underside of the lower part (2), the body has recesses.

6. The device according to claim 5, characterized in that on the undersigned of the lower part (2), the body has self-locking pads (3).

7. The device according to claim 6, characterized in that the self-locking pads (3) are silicone inserts having thickness of 1 mm to 3 mm.

8. The device according to claim 1, characterized in that in the side wall of the arched upper part (1), the body has a hole.

9-12. (canceled)
